(19) United States
(12) Patent Application Publication
(10) Pub. No.: US 2010/0152659 A1
(43) Pub. Date: Jun. 17, 2010

(54) INJECTION DEVICE WITH ANTI-TRIGGER LOCKS

(76) Inventors: Ursina Streit, Schoenbuehl (CH); Markus Bollenbach, Bern (CH); Ulrich Moser, Heimiswil (CH); Juerg Hirschel, Aarau (CH); Celine Kaenel, Oberburg (CH); Daniel Kuenzli, Langendorf (CH); Markus Tschirren, Kirchberg (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/563,689

(22) Filed: Sep. 21, 2009

Related U.S. Application Data
(63) Continuation of application No. PCT/EP2008/053510, filed on Mar. 25, 2008.

(30) Foreign Application Priority Data
Mar. 22, 2007 (DE) 10 2007 013 836.7
Mar. 22, 2007 (DE) 10 2007 013 837.9
Mar. 22, 2007 (DE) 10 2007 013 838.7

(51) Int. Cl. A61M 5/32; A61M 5/20

(52) U.S. Cl. 604/136; 604/192

(57) ABSTRACT

A device for administering a medicinal substance, the device including a first element and a second element, wherein at least one of the first and second elements is moveable relative to the other, a lock element moveable to and from a locked position in which it prevents the relative movement of the at least one of the first and second elements and an unlocked position in which it permits the relative movement of the at least one of the first and second elements, and a safety element moveable to and from a secured position in which it prevents the lock element from moving from the locked position to the unlocked position and an unsecured position in which it permits the movement of the lock element to the unlocked position.

### CROSS-REFERENCED RELATED APPLICATIONS

This application is a continuation of International Patent Application No. PCT/EP2008/053510 filed Mar. 25, 2008, which claims priority to German Patent Application No. DE 10 2007 013 838.8 filed Mar. 22, 2007, German Patent Application No. DE 10 2007 013 836.0 filed Mar. 22, 2007 and Germany Patent Application No. DE 10 2007 013 837.9 filed Mar. 22, 2007, the entire contents of each are incorporated herein by reference.

### BACKGROUND

The present invention relates to devices for injecting, infusing, administering, delivering or dispensing a substance, and to methods of making and using such devices. More particularly, it relates to an injection device for administering a substance or a product, e.g., an automatic injector. The substance or product may be a liquid medicament, such as insulin, growth hormone, etc., for example. In some embodiments, the injection device may be designed so that a manual piercing by a needle and automatic dispensing of the product is possible. In some embodiments, the injection device may take the form of an automatic injector which, when triggered, provides for automatic piercing by the needle and automatic dispensing of the product. In some embodiments, the automatic injector may cause the needle to retract automatically after the injection or, in some embodiments, the needle may be removed manually.

Injection devices are known from the prior art and, of course, contain parts that can be moved relative to one another. These parts may be locked relative to one another by a lock or lock element, in which case releasing the lock or lock element permits a movement of the two parts relative to one another. During transport or if handling the injection device, when a relative movement of the two parts with respect to one another should be avoided, vibrations, inadvertently applied forces, etc., can cause the lock between the two parts to release. This can have negative consequences, such as unintentionally triggered dispensing of a product, for example.

### SUMMARY

One object of the present invention is to provide an injection device with improved reliability.

In one embodiment, the present invention comprises a device for administering a medicinal substance, the device comprising a first element and a second element, wherein at least one of the first and second elements is moveable relative to the other, a lock element moveable to and from a locked position in which it prevents the relative movement of the at least one of the first and second elements and an unlocked position in which it permits the relative movement of the at least one of the first and second elements, and a safety element moveable to and from a secured position in which it prevents the lock element from moving from the locked position to the unlocked position and an unsecured position in which it permits the movement of the lock element to the unlocked position.

In one embodiment, the present invention comprises a device for administering a product, e.g., a medicament, comprising a first element, a second element which can be moved relative to the first element, a lock element, which can be moved from a locked position, in which it prevents a movement between the first element and the second element, into an unlocked position, in which it permits the movement between the first element and the second element, and a safety element which can be moved from a secured position, in which it blocks the lock element to prevent the movement thereof out of the locked position into the unlocked position, into an unsecured position, in which it permits the movement of the lock element into the unlocked position.

In some embodiments, the present invention comprises a device for administering a product, e.g. an injection device, e.g. an automatic injector. The device comprises a first element and a second element able to move relative to the first element. The elements may rotatably and/or axially displaceable, or can effect a combination of these movements. The first and second elements may rest one against the other or may slide one on the other relative to one another during a movement. In some embodiments, the first and second elements are at least adjacent. For example, the first element and/or the second element may be sleeve-shaped, in which case one of the first element and second element may be arranged inside the other of the first element and second element. The two elements may be arranged concentrically with respect to one another. A sleeve shape need not necessarily be a closed tubular shape, and may incorporate one or more apertures and/or ribs, for example.

In some embodiments, the injection device comprises a lock element which can be moved from a locked position, in which it prevents a movement between the first element and the second element, to an unlocked position, in which it releases at least one of the first and second elements for relative movement. The lock element may be a separate part, such as a cam, bolt or sphere for example, or may be disposed on one of the first element and second element, e.g., is an integrally formed part. For example, the lock element may be elastically mounted, for example disposed on the first element or second element by an elastic arm. In some embodiments, the lock element is disposed so that it tends to move out of the locked engagement or remain in the locked engagement and, in some preferred embodiments, the lock element can be moved out of the locked engagement at the latest during a relative displacement between the first element and second element. To this end, the shape of the lock element may be such that it is pushed out of the locked engagement when there is a relative displacement between the first element and second element, thereby assuming the unlocked position.

In some embodiments, the lock element can be moved from a locked position to an unlocked position by a movement directed transversely to the longitudinal length or extent of the injection device. Such a movement may be a movement directed in the circumferential direction, for example, or a movement in the radial direction, for example a movement directed towards a mid-axis or longitudinal axis of the injection device or directed away from the mid-axis or longitudinal axis. The movement may be a linear movement or a pivoting or rotating movement.

In some preferred embodiments, a device in accordance with the present invention comprises a safety element which can be moved from a locked position, in which it prevents the lock element from moving, e.g., out of a locked position to an unlocked position, into an unlocked position, in which it releases or permits the movement of the lock element into the unlocked position. In the secured position, the safety element is able to hold the lock element in its locked position so that a movement between the first element and the second element is blocked by the lock element. If, for example, a force that would cause a relative movement between the first element and second element is applied to the first element or to the second element, no relative movement is able to take place between the first element and the second element because the safety element prevents a movement of the lock element. This can be applicable in situations where the lock element tends to move out of the locked engagement, for example due to a spring force, and also in situations in which the lock element is forced out of the locked engagement due to the relative movement between the first element and the second element. A relative movement can be reliably prevented between the first element and the second element as a result of such an arrangement, no matter what type of lock or lock element is employed. The resultant advantages are that lock elements in an injection device can be relatively weak or e.g., a filigree design, thereby reducing the size of the injection device and increasing the reliability of the injection device. A buyer or user will find the injection device more appealing because it will seem less bulky.

In some embodiments, to effect a relative movement, the first element and the second element may be biased by a pre-tensioned spring, for example, so that a relative movement would take place between the two elements without the lock element. In the case of injection devices and automatic injectors which can be pre-tensioned with a relatively strong force to dispense the product or for the piercing action, the lock elements used must be of a relatively strong design to enable the lock element to remain in the locked engagement in spite of the relatively high force. This also requires stronger switching forces, i.e. the forces needed to release the locked engagement. By using a safety element in accordance with the present invention, the lock element can be of a filigree-type design because it reliably holds the lock element in the locked engagement when in the secured position. The switching forces can also be reduced. In the locked engagement or in the locked position, the lock element engages in at least one of the first or second element.

In the secured position, the safety element blocks the lock element and prevents it from moving from the locked position to the unlocked position. Hereto, the safety element is arranged in the direction of movement in which the lock element is able to move once the safety element has been moved out of the secured position. For example, the safety element is able to effect a rotating movement or an axial movement, by which it can be moved out of the secured position into the unsecured position. The rotating movement may be directed about a longitudinal axis, for example, in the forward-driving or injection direction of a product container inserted in the device or in the dispensing or delivery direction of a product. Alternatively or in addition, the safety element may effect an axial movement, for example transversely, but directed along the longitudinal axis, when it is moved out of the secured position into the unsecured position. The safety element may be disposed on the same level as the lock element—as viewed along the longitudinal axis or in a longitudinal direction. Alternatively or in addition, the safety element may assume a position of angular rotation directed about the longitudinal axis or longitudinal direction relative to the lock element so that the safety element is rotated to an angular position in front of the lock element. For example, the safety element is able to assume different positions of angular rotation, e.g. a secured position and an unsecured position.

For example, during the movement out of the secured position into the unsecured position, the safety element may move in the same direction as the lock element when it moves out of the locked position into the unlocked position. In some preferred embodiments, when moving from the secured position into the unsecured position, the safety element moves in the direction extending transversely to that in which the lock element is able to move from the locked position into the unlocked position.

During the movement of the lock element into the unlocked position, it may be moved toward the longitudinal axis of the injection device or away from the longitudinal axis of the injection device. For example, as viewed from the longitudinal axis or mid-axis of an injection device in the radial direction, the second element may be disposed inside the first element or vice versa. The safety element may also be arranged inside of the first element or second element. For example, the second element may be arranged inside of the first element and the safety element may be arranged inside of the second element and hence also inside of the first element.

In some embodiments, the safety element can be removed from the injection device in the unsecured position, i.e. physically separated from it, or may be left on or in the injection device, i.e. connected to the injection device.

In some embodiments, the locking arrangement in accordance with the present invention may be disposed in a plurality of positions in an injection device. For example, the lock arrangement of the present invention may be fitted wherever a specific device such as a driving member or plunger rod has to co-operate in switching operations depending on position. It is possible to obtain a sequence control for example, in which case a switch can be made between a piercing movement and a dispensing movement in the case of an automatic injector. A locking arrangement in accord with the present invention can also be used wherever one or more parts are moved to trigger dispensing of a product. In this respect, it is preferable to prevent any unintentional movement of such parts caused by, for example, mass inertia if dropped or vibrations during transport. In some embodiments, the arrangement can be disposed on a trigger mechanism or integrated in or with the trigger mechanism, in which case the negative consequences of force being inadvertently applied to the injection from outside or due to an “accident” can be avoided. The injection device is safer as a result.

In some embodiments, a lock or locking arrangement in accordance with the present invention may be disposed in a device for administering a product which, for example, may have an operating element such as an operating sleeve. When the operating element is operated, a product dispensing operation or piercing operation can be initiated indirectly or directly. In some embodiments, the operating element may be regarded as the second element and may be mounted on a housing of the device, which may be regarded as the first element. When the operating element is operated, it is moved relative to the housing, e.g. axially displaced. For example, the housing and the operating element may be sleeve-shaped and/or cylindrical. The operating element may extend out from the device laterally, e.g. transversely to the longitudinal direction, or axially, i.e. in the longitudinal direction. For example, the operating element may extend from the device in the proximal (rearward) direction, so that it can be operated by a user's thumb, or in the distal (forward or injection) direction, so that it can be operated by applying it to an injection site.

In some preferred embodiments, the operating element extends distally beyond the distal end of the housing. The distal end of the operating element may be placed on an injection site of a patient. The user grips the housing and presses the housing in the direction of the patient or injection site. As a result, the operating element is displaced relative to the housing, e.g. is pushed into the housing. This enables a product dispensing operation or a piercing operation to be triggered. To prevent a product dispensing operation from being inadvertently triggered, the operating element and/or the distal end of the housing may be covered by a cover, e.g. a cap. The cap is able to protect the operating element against inadvertent operation because it prevents access to the operating element. In spite of the fact that a cap is fitted, situations can occur with conventional devices in which, if the device is dropped on the floor for example, the operating element can be displaced due to mass inertia, which can lead to undesired triggering of the device. This problem can be avoided in the case of a device incorporating the present invention. For example, one of the housing and operating element may have at least one lock means, which engages in the other of the housing and operating element. For example, an element in which the lock element engages may have a recess for the lock element. In principle, it is sufficient if the lock element locates in the front face of the housing. As long as the lock element is in its locked position, i.e. engaged, it is able to prevent a movement between the operating element and the housing, although it would in theory be possible for the lock element to be moved out of engagement by a movement of the operating element relative to the housing, i.e. from the locked position into an unlocked position. In some preferred embodiments, the lock element may be disposed on a resilient arm on the operating element and moved more or less in the radial direction.

In some embodiments, to prevent the lock element from being pushed out of engagement with the housing due to a movement of the operating element, a safety element in accordance with the present invention is provided, which is able to assume a secured position in which it blocks the lock element and prevents the movement out of the locked position into the unlocked position. The safety element can be moved out of the secured position into an unsecured position, thereby enabling a movement of the lock element into the unlocked position. For transport purposes or in the state in which the injection device is supplied, the safety element may be disposed in the secured position. The safety element may be a part or disposed on a part which is removed before using the injection device. In some preferred embodiments, the safety element is provided in the form of the cap. Accordingly, when the cap is fitted, the cap may have a surface which is disposed in front of the lock element in the direction in which the lock element is moved so that the lock element can not be moved. In some embodiments, the surface may be disposed so that it holds the lock element in the locked position or pushes it into the locked position, i.e. into engagement. Removing the cap corresponds to the movement of the safety element out of the secured position into the unlocked position because the surface releases the movement of the lock element from the locked position. In some preferred embodiments, the surface preventing the lock element from moving out of the locked position is arranged inside the sleeve-shaped operating element in the secured position. The operating element can be pushed in the distal direction by a spring, e.g. a return spring, so that when the operating element is operated in the direction opposite the spring force, the return spring is tensed. In the non-operated state, this means that the operating element extends beyond the housing in the distal direction as far as necessary to enable the device to be triggered when the sleeve is pushed back by the distance of this over-extension.

In some embodiments, the arrangement of the present invention may also be provided where a drive member is mounted and released to effect a driving movement. The drive member may correspond to the second element and may be a function sleeve or a plunger rod, for example. The driving movement may be used to enable the needle to effect a piercing movement and/or to dispense the product, for example. The driving movement may be caused by a pre-tensioned driving spring, for example, if the second element can be moved relative to the first element. The first element may be a housing or an element connected to the operating element, e.g. in an axially fixed arrangement, such as a switch sleeve, for example. The first element may be arranged at the operating element.

In some embodiments, the safety element may be coupled with or connected to the first element in an axially fixed arrangement, for example to the housing or the switch sleeve. The safety element is able to effect a rotating movement to move out of the secured position into the unsecured position. The movement may be effected by the safety element before triggering a dispensing or piercing operation to “activate” the injection device prior to a triggering movement, or during it to “activate” it during a triggering movement. For example, at least one position lock may be provided, which is mounted on the safety element so that it is able to move and which causes the safety element to be held in the secured position or/and the unsecured position so that the safety element initially has to overcome increased resistance as it is being moved out of the secured position. This resistance is higher than the resistance which has to be overcome during the transition from static to sliding friction. In some preferred embodiments, the position lock is based on a positive or/and non-positive engagement of the safety element in the element to which the safety element is secured, for example a positive connection which can be released by a force or torque. The force or torque may be directed in a direction corresponding to the subsequent direction of movement needed to bring about the release. When the safety element is in the unsecured position, a position lock causes the safety element to remain in the unsecured position on reaching it or at least ensures that it cannot be moved out of the unsecured position inadvertently, i.e. without further action. The position lock may be a cam or, more generally, a suitable structure or projection, which engages in a notch or, more generally, a recess or complementary structure. The position lock is released or unlocked due to the elasticity of the material of the safety element or/and the part accommodating the safety element which co-operates in the action of locking the position.

In some embodiments, during the triggering movement, the safety element may be rotatable, e.g. manually or automatically. In the case of a safety element which is manually rotated, which is moved into the unsecured position prior to the triggering movement, the safety element may be provided in the form of a knob on the housing which can be gripped by the user or may at least be provided on the knob. In addition, an activator cam may be provided on the housing or rotatable knob, which is able to move the lock element out of the locked engagement. The activator cam may be angularly offset from the activator lock in the circumferential direction of the device so that either the activator lock or the activator cam is selectively in an angular position of the knob matching that of the lock element depending on the position of rotation of the knob.

In some preferred embodiments, a safety element which can be released automatically is protected to prevent access by the user or is disposed inside the housing, for example.

In some embodiments, a guide or gear element may be provided, which converts the operating movement of the operating element into a movement of the safety element out of the secured position into the unsecured position. The gear element may be provided on the housing or in the form of an element secured to the housing or the switch sleeve or another element secured to the operating element. Generally speaking, the gear element is disposed so that a relative movement takes place between the safety element and the gear element during a triggering movement of the operating element. This relative movement may be directed along the longitudinal axis of the injection device, e.g. in a proximal (rearward) direction. Due to the relative movement, the safety element can be moved out of the secured position by a rotating movement. For example, the gear element may be disposed in a stationary arrangement relative to the housing, prevented from rotating and moving axially, and the safety element may be disposed so that it is not able to move axially but can rotate relative to the operating element. Or the gear element is stationary with respect to the operating element, is not able to rotate and move axially, and the safety element is disposed so that it is not able to move axially but can rotate relative to the housing. The lock element may be axially stationary relative to the safety element during operation, e.g. if the safety element is disposed on the operating element so as to move axially with it, or may be displaceable, e.g. if the safety element is disposed on the housing so that it is able to move axially with it.

In some embodiments, e.g. in the case of a safety element which is moved axially out of the secured position, it may be coupled with the operating element in an axially fixed arrangement. The safety element is moved out of an axial position in which it sits or rests on a level with the lock element into an axial position in which it no longer sits or rests on a level with the lock element, as a result of which there is no longer any support for the lock element. In some embodiments, the safety element is disposed proximally of the lock element in its unsecured position.

The present invention encompasses method steps which will become apparent from the operating mode of a device in accordance with the present invention. It should be appreciated that method steps involving the device may be implemented or take place without using the device to administer to a patient.

### BRIEF DESCRIPTION OF THE DRAWINGS

FIGS. 1a and 1b are sectional diagrams of one embodiment of an injection device in accordance with the present invention, fitted with a cap, FIG. 1b being a view rotated 90° about the longitudinal axis compared with FIG. 1a.

FIGS. 2a and 2b are sectional diagrams showing the injection device illustrated in FIGS. 1a and 1b with the cap removed, FIG. 2b showing a view rotated 90° about the longitudinal axis compared with FIG. 2a.

FIGS. 3a and 3b are sectional diagrams showing the injection device illustrated in FIGS. 1a and 1b in an activated state, FIG. 3b showing a view rotated by 90° about the longitudinal axis compared with FIG. 3a.

FIGS. 4a and 4b are sectional diagrams showing the injection device illustrated in FIGS. 1a and 1b in a triggered state, FIG. 4b showing a view rotated by 90° about the longitudinal axis compared with FIG. 4a.

FIGS. 5a and 5b are sectional diagrams showing the injection device illustrated in FIGS. 1a and 1b in a piercing state, FIG. 5b showing a view rotated by 90° about the longitudinal axis compared with FIG. 5a.

FIGS. 6a and 6b are sectional diagrams showing the injection device illustrated in FIGS. 1a and 1b in a dispensed state, FIG. 6b showing a view rotated 90° about the longitudinal axis compared with FIG. 6a.

FIGS. 7a and 7b are sectional diagrams showing the injection device illustrated in FIGS. 1a and 1b in a state in which the injection device has emitted a clicking noise to signal the end of dispensing, FIG. 7b showing a view rotated 90° about the longitudinal axis compared with FIG. 7a.

FIGS. 8a and 8b are sectional diagrams showing the injection device illustrated in FIGS. 1a and 1b in a situation in which retraction of the injection needle has been activated, FIG. 8b showing a view rotated 90° about the longitudinal axis compared with FIG. 8a.

FIGS. 9a and 9b are sectional diagrams showing the injection device illustrated in FIGS. 1a and 1b in a final state, FIG. 9b showing a view rotated 90° about the longitudinal axis compared with FIG. 9a.

FIGS. 10a and 10b are sectional diagrams of another embodiment of an injection device in accordance with the present invention, FIG. 10b showing a view rotated 90° about the longitudinal axis compared with FIG. 10a.

FIGS. 11a and 11b are sectional diagrams showing the injection device illustrated in FIGS. 10a and 10b with a cap removed, FIG. 11b showing a view rotated 90° about the longitudinal axis compared with FIG. 11a.

FIGS. 12a and 12b are sectional diagrams showing the injection device illustrated in FIGS. 10a and 10b in a triggered state, FIG. 12b showing a view rotated 90° about the longitudinal axis compared with FIG. 12a.

FIG. 13 is a perspective view of the safety element illustrated in FIGS. 10a and 10b disposed in a secured position.

FIG. 14 is a perspective view of the safety element illustrated in FIG. 13, moved into an unsecured position.

FIG. 15 is a perspective view of a separately provided safety element.

FIG. 16 is a perspective view of a switch sleeve for the device illustrated in FIGS. 10a and 10b.

FIG. 17 is a sectional diagram of another embodiment of an injection device in accordance with the present invention with a safety element in a secured position,

FIG. 18 is a sectional diagram showing the injection device illustrated in FIG. 17 with the safety element in an unsecured position.

### DETAILED DESCRIPTION

With regard to fastening, mounting, attaching or connecting components of the present invention, unless specifically described as otherwise, conventional mechanical fasteners and methods may be used. Other appropriate fastening or attachment methods include adhesives, welding and soldering, the latter particularly with regard to the electrical system of the invention, if any. In embodiments with electrical features or components, suitable electrical components and circuitry, wires, wireless components, chips, boards, microprocessors, inputs, outputs, displays, control components, etc. may be used. Generally, unless otherwise indicated, the materials for making embodiments of the invention and/or components thereof may be selected from appropriate materials such as metal, metallic alloys, ceramics, plastics, etc.

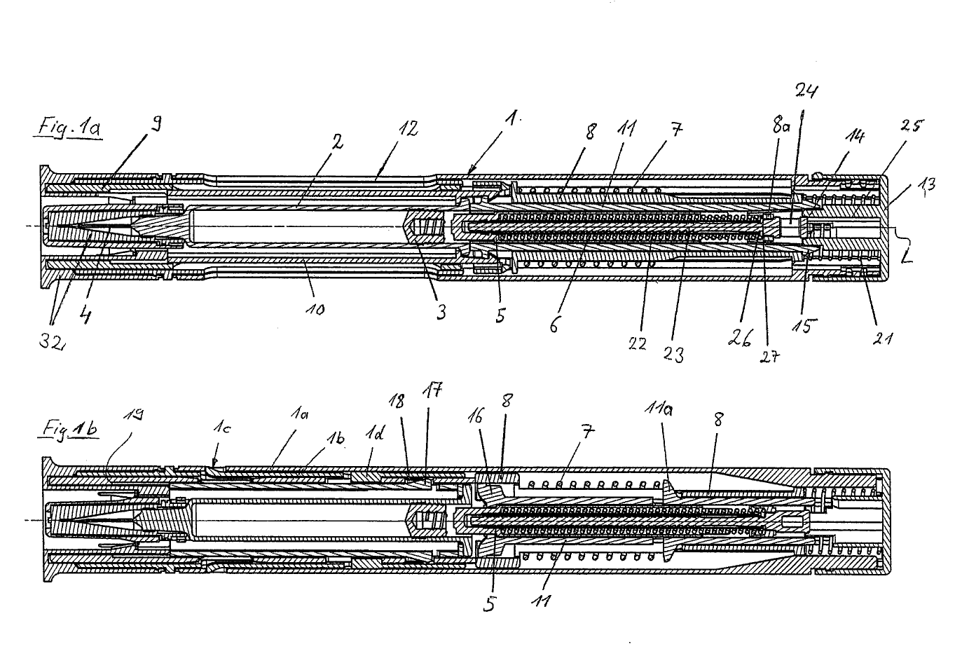

The accompanying Figures illustrate embodiments of the present invention, including embodiments of an injection device in accordance with the present invention. Referring to FIGS. 1a and 1b, the injection device comprises a housing 1, with a proximal housing part 1a and a distal housing part 1b connected to the proximal housing part by a catch connection 1c so as to be axially fixed. The catch connection 1c comprises a window associated with the proximal housing part and an elastic tongue associated with the distal housing part 1b. The tongue snaps into the window.

A product container 2 is accommodated in the housing. On the distal (front or forward) end of the container is an injection needle 4 for dispensing a liquid product contained in the product container 2. At the proximal end, the product container 2 has a displaceable plunger 3, the movement of which relative to the product container 2 and in the direction of the injection needle 4 causes product to be dispensed; the movement may be thought of and referred to as a dispensing or administering movement. The product container 2 is accommodated in the device so that it is able to move in the distal direction so that the injection needle 4 extends out beyond the distal end of the injection device. This may be thought of and referred to as a piercing movement. The product container 2 is connected to a holder 10 for holding the product container 2 in an axially fixed arrangement. The housing 1, e.g. its distal and proximal housing parts 1a, 1b, have a viewing window 12 through which the user of the injection device can see the product container 2. The holder 10 surrounds the product container 2 in a sleeve shape and either has a viewing window itself or, as in this example, is made from a transparent material to expose the container 2. The product container 2 is connected in an axially fixed arrangement by a clamp to a function sleeve 11 disposed proximally (rearwardly) of it at the proximal (rear) end of the holder 10. At its proximal end, the product container 2 has a radially projecting collar, which is gripped by the clamp. At its distal end, the function sleeve 11 likewise has a radially projecting collar, which is also enclosed by the clamp. Accordingly, the product container 2, function sleeve 11 and holder 10 are connected to one another in an axially fixed arrangement so that they are able to move as a single part. This combination may be thought of and referred to herein as a drive structure.

The function sleeve 11 surrounds a plunger rod 5 able to act on the plunger 3 to dispense product. The plunger rod 5 has a sleeve-shaped part which surrounds a driving spring 6. The driving spring 6 is supported distally on the plunger rod 5 and proximally on a switch sleeve 8, being operably coupled to a socket 8a.

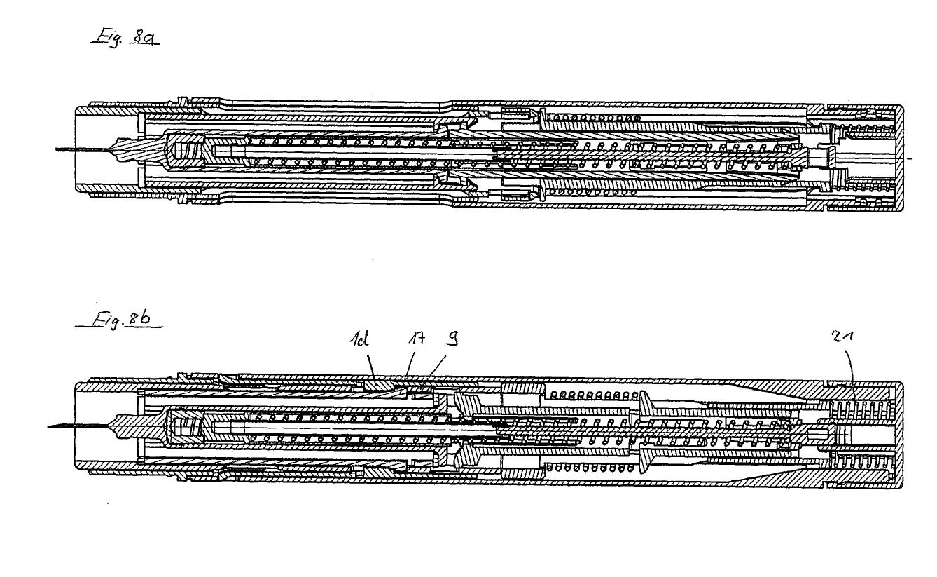

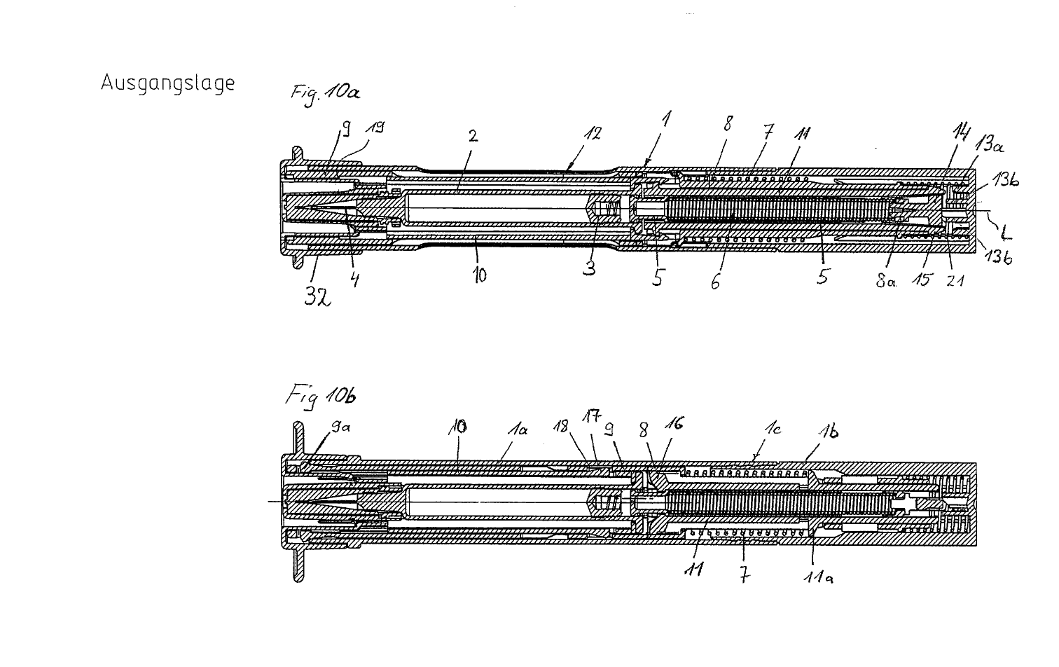

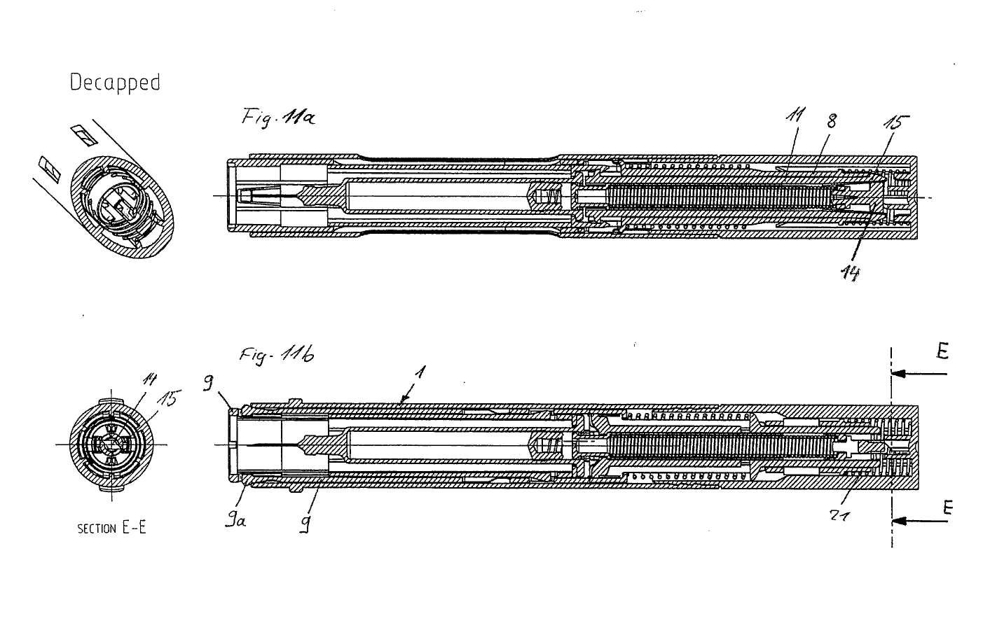

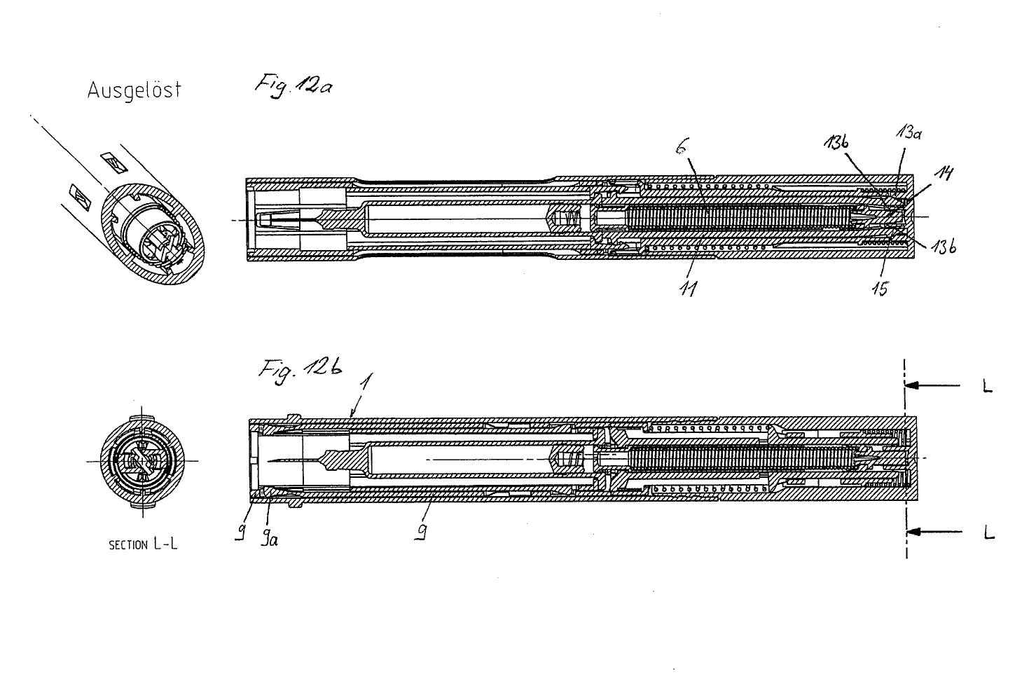

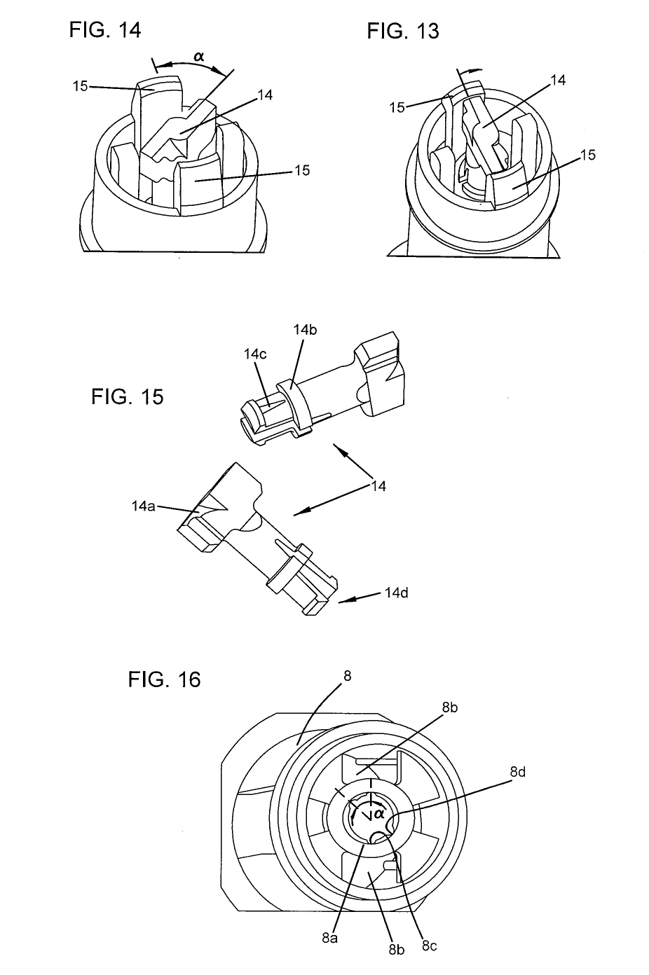

Adjoining the plunger rod 5 is a signalling unit, by which one or at least three or more haptic and/or acoustic signals can be generated for the piercing operation and/or the dispensing operation. The signalling unit comprises a catch rod 23 connected to the switch sleeve 8 and a locating sleeve 22 surrounding the catch rod 23 and connected, e.g. latched, in an axially fixed arrangement to the plunger rod 5. The locating sleeve 22 has a locating element 26 which engages in a groove 27 of the catch rod 23. At its proximal end, the catch rod 23 has a head 24, which is able to move in the proximal direction in a slide guide 25 formed by the activator element 13. The head engages by its distal end with a socket 8a disposed on the switch sleeve 8 and the engagement prevents the head 24 and hence the catch rod 23 from being able to move relative to the switch sleeve 8 in the distal direction. The way this arrangement operates will be explained further later with reference to FIGS. 10 and 11, which provide a detailed illustration of the signalling unit illustrated in FIGS. 1 to 9. Alternatively, the signalling unit illustrated in FIGS. 10 and 11 may be replaced by a different signalling unit illustrated in FIGS. 12 to 14 and by yet another signalling unit illustrated in FIGS. 15 and 16. The injection devices illustrated in FIGS. 1 to 9 do not have to undergo any major modifications to this end.

When the injection device is in the initial state illustrated in FIGS. 1a and 1b, the driving spring 6 is tensed so that the needle 4 and the drive structure are advanced forward for a piercing movement and can push the plunger 3 to effect a dispensing or administering movement. The function sleeve 11 has a lock element 16, on which a shoulder is disposed and directed radially inwardly. In the initial state, the shoulder co-operates with another shoulder directed radially outwardly on the distal end of the plunger rod 5 so that the plunger rod 5 is locked, thereby preventing a movement relative to the function sleeve 11. The lock element 16 is held in engagement with the plunger rod 5 by a surface of the switch sleeve 8 pointing radially inwardly. In some embodiments, the lock element 16 is elastically connected to the function sleeve 11 by a resilient arm. The resilient arrangement may be designed so that the lock element 16 tends to move radially outward, but this is prevented by the surface of the switch sleeve 8 pointing radially inward.

At its proximal end, the function sleeve 11, which, in some embodiments, may be thought of and referred to as the second element within the context of the invention, has at least one snapper element 15, which snaps into the switch sleeve 8 in the initial state to prevent any movement of the function sleeve 11 and hence the drive structure. As a result, the pre-tensed spring 6 is not yet able to relax and the drive structure is not yet able to move in the distal direction.

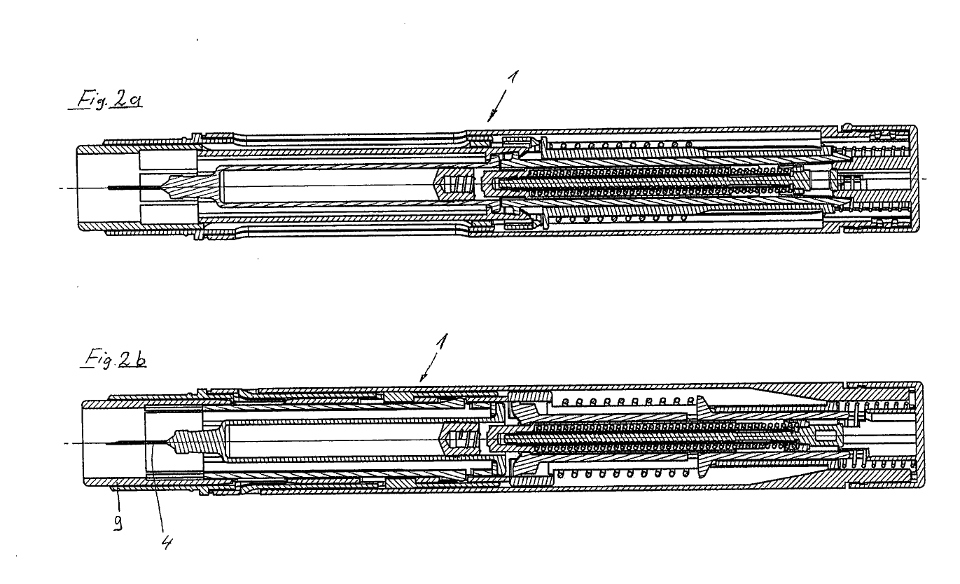

At the proximal end of its housing 1, the injection device has an activator element 13, which is disposed so that it is axially stationary, but can be rotated relative to the housing 1. The activator element 13 houses a return spring 21, which is supported distally on the proximal end of the switch sleeve 8 and proximally on the activator element 13. The purpose of the return spring 21 is to apply a force acting in the distal direction to the switch sleeve 8 and an operating sleeve 9 acting axially on the switch sleeve 8 so that the switch sleeve 8 and operating sleeve 9 are moved in the distal direction. The activator element 13 has an activator lock 14, which engages behind the snapper element 15 when the injection device is in the switching states illustrated in FIGS. 1a, 1b, 2a and 2b so that the snapper element 15 is blocked or locked and is not able to move out of engagement with the switch sleeve 8. This advantageously prevents the injection device from being inadvertently triggered. The user has access to the activator element 13, and thus, in a broader sense, to the trigger lock. The activator lock can be moved out of engagement with the snapper element 15 by turning the activator element 13 by 90° relative to the housing 1, for example.

A return spring 7 acting along the longitudinal length or axis of the device is distally supported on the switch sleeve 8 and proximally supported on the function sleeve 11. As illustrated in this example, the return spring 7 surrounds the switch sleeve 8 and the function sleeve 11. The return spring 7 is proximally supported on a collar 11a disposed on the function sleeve 11, which extends radially outward through an aperture provided in the switch sleeve 8. In specific switch positions therefore, the return spring 7 is able to cause a relative movement between the switch sleeve 8 and function sleeve 11. The return spring 7 is a compression spring which is able to move the function sleeve 11 in the proximal direction relative to the switch sleeve 8. The return spring 7 is not pre-tensioned or pre-tensioned with only a slight pre-tensioning force. For example, when the injection device is in the state illustrated in FIGS. 1a and 1b, the pre-tensioning force of the return spring 7 is lower than the pre-tensioning force of the driving spring 6.

Disposed distally of the switch sleeve 8 is the operating sleeve 9 which is able to move relative to the housing 1. The switch sleeve 8 and the operating sleeve 9 are mutually able to apply a pressing force to one another, e.g. latch with one another, thereby pushing one another. To prevent the view of the product container 2 being blocked by the operating sleeve 9, the operating sleeve 9 also has a window in the region of the window 12. Alternatively, the operating sleeve 9 may be made from a transparent material. When the return spring 21 is in the initial state, the operating sleeve 9 is pushed forward by the return spring 21 via the switch sleeve 8 distally beyond the distal end of the housing 1. The distal end of the operating sleeve 9 is used for positioning on an injection site of a patient.

The holder 10 has a switch cam 17, which engages in a cut-out 18 of the operating sleeve 9, which may be provided in the form of an aperture as illustrated in this example. The switch cam 17 is elastically connected, in some embodiments integrally, to the holder 10 by a resilient arm, for example. The switch cam 17 is biased so that it tends to engage in the cut-out 18 or move radially outwardly. The switch cam 17 projecting radially outward from the holder 10 has an oblique surface distally, which therefore also co-operates in pushing the switch cam 17 out of engagement with the cut-out 18. Proximally, the switch cam 17 also has a transversely extending stop surface, e.g. perpendicular to the longitudinal direction, able to make axial contact with the proximal boundary of the cut-out 18, as a result of which the switch cam 17 is not able to be moved out of the cut-out 18.

The operating sleeve 9 has an axial stop 19, with which the distal end of the holder 10 is able to make contact at the end of the piercing movement.

As illustrated in FIGS. 1a and 1b, the distal end of the injection device is fitted with a cap 32, which protects the interior of the injection device from dirt and keeps the needle 4 sterile. The cap 32 is removed prior to using the injection device so that the needle 4 and the operating sleeve 9 are exposed, as illustrated in FIGS. 2a and 2b. The state of the injection device illustrated in FIGS. 2a and 2b differs from the state illustrated in FIGS. 1a and 1b due to the fact that the cap 32 has been removed.

The force exerted on the injection device when the needle cap 32 is pulled off is transmitted via the holder 10 to the function sleeve 11, from where it is transmitted via the snapper 15 to the switch sleeve 8, which is supported on the operating sleeve 9. The operating sleeve 9 is in turn latched to the housing 1 via a projection 1d disposed on the distal housing part 1 so that the action of pulling the cap 32 off the injection device does not have any undesired effect on the mechanism.

In the switch state illustrated in FIGS. 2a and 2b, the operating sleeve 9 can not or can only very slightly be pushed into the distal end of the injection device because this sliding movement is transmitted via the switch sleeve 8 to the snapper 15 and the snapper 15 is prevented from moving in the proximal direction by the activator element 13.

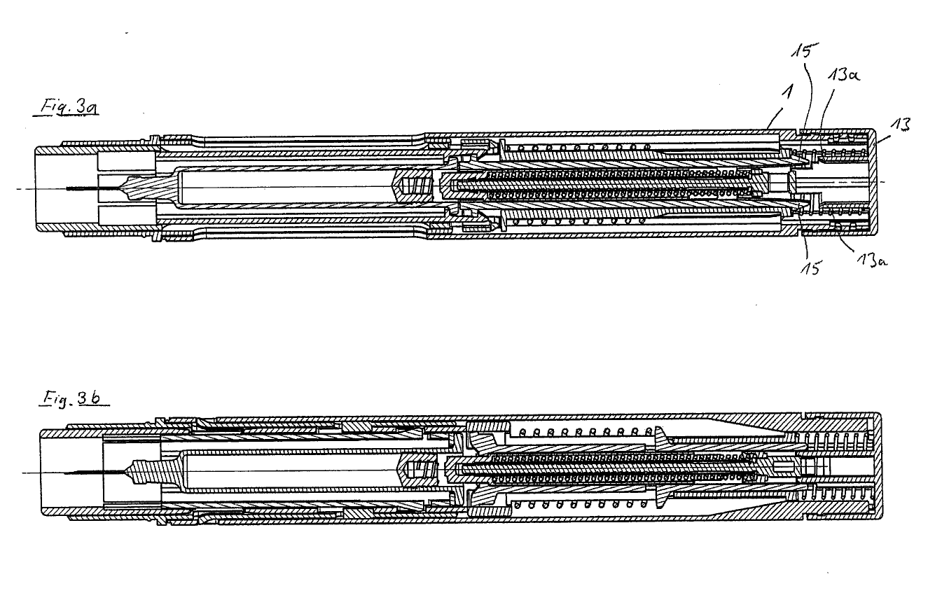

The injection device is illustrated in an activated state in FIGS. 3a and 3b, i.e. the injection device can be triggered. The injection device is activated or unlocked by a rotating movement of the activator element 13, e.g. by 90°. As this happens, the snapper elements 15 are released to permit a movement directed radially inwardly due to the fact that the activator lock 14 is moved, e.g. rotated, out of engagement with the snapper elements 15. Consequently, there is space for the snapper elements 15 to be deflected inwardly. Like the snapper element 15, the activator element 13 has an activator cam 13a, which is moved into a position axially flush with the snapper element 15 by the rotating movement of the activator element 13. Proximally, the snapper element 15 and, distally, the activator cam 13a disposed proximally of it, have a contour which can deflect the snapper element 15 radially inwardly as the snapper element 15 moves into engagement with the activator cam 13. In this example, the contours are two oblique planes extending toward one another.

To trigger the injection device, the user places the device, which has previously been activated by rotating the activator lock 14, with the distal end on the injection site which has been disinfected beforehand. As a result, the operating sleeve 9 is moved in the proximal direction relative to the housing 1 until the distal end of the operating sleeve 9 is more or less flush with the distal end of the distal housing part 1b. Due to the movement of the operating sleeve 9, the switch sleeve 8 is also slaved in the proximal direction, causing the snappers 15 to be pushed radially inwardly by the activator cam 13a and out of engagement with the switch sleeve 8. As the operating sleeve 9 moves in the distal direction, the elements of the drive structure (comprising elements 2, 10, 11) are also moved in the proximal direction, as long as the snapper elements 15 are snapped into the switch sleeve 8. Since the plunger rod 5 is in a locked engagement with the function sleeve 11, the plunger rod 5 is also moved in the proximal direction. The signalling unit accommodated in the plunger rod 5 is likewise moved in the proximal direction. The head 24 disposed proximally on the catch rod 23 is able to slide along the guide 25 formed by the activator element 13.

Since no relative movement can yet take place between the activator sleeve 11 and the switch sleeve 8 during this movement, neither the return spring 7 nor the driving spring 6 are tensed or relaxed.

The force which the user of the device must apply to the housing 1 to push the operating sleeve 9 in the proximal direction is essentially determined by the force of the return spring 21 against which the switch sleeve 8 and the operating sleeve 9 are moved. In some embodiments, the spring 21 is a compression spring and is made from a plastic material. Alternatively, it would be possible to use springs made from spring steel material or some other spring material. The activator element 13 is axially secured to the housing 1 by a ring snapper connection to the housing. If the operating sleeve 9 is not pushed far enough toward the injection site and the snapper elements 15 are not released from the engagement with the switch sleeve 8, the trigger mechanism, e.g. the switch sleeve 8 and the operating sleeve 9, are re-set by the return spring 21 when the injection device is moved away from the injection site.

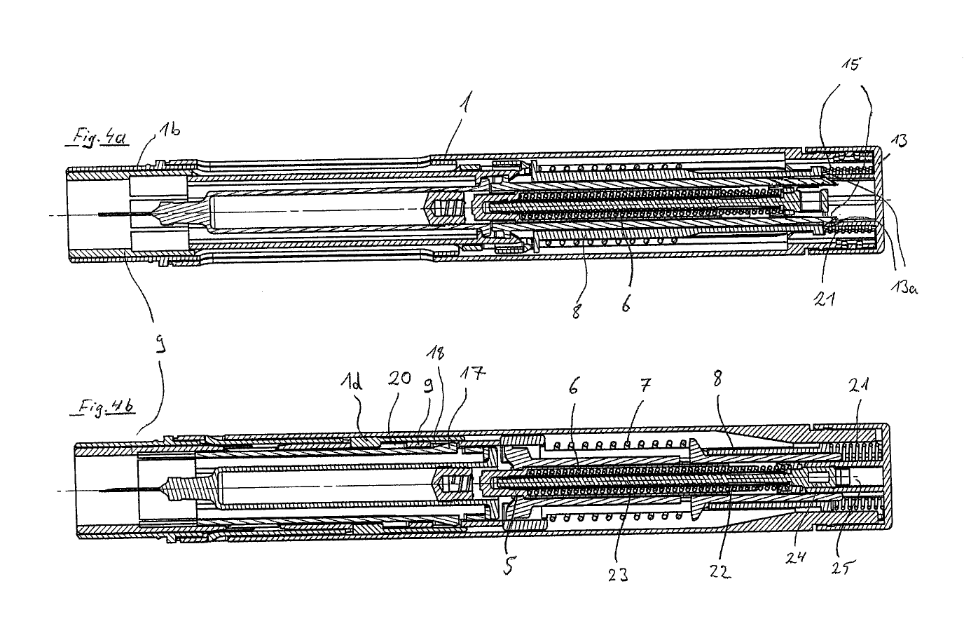

As may be seen from FIG. 4b, a lock window 20 is formed due to the movement of the operating sleeve 9 in the proximal direction, which is bounded distally by the housing 1, e.g. the projection 1d, and proximally by the operating sleeve 9. Since no relative movement can yet take place between the drive structure and the operating sleeve 9 as the operating sleeve 9 is moving in the proximal direction, the switch cam 17 remains in the cut-out 18.

Once the snappers 15 have been released from the engagement with the switch sleeve 8, the driving spring 6 is able to relax to a certain extent, as a result of which the drive structure is pushed in the distal direction. This being the case, the injection needle 4 moves beyond the distal end of the injection device. Since the function sleeve 11 moves relative to the switch sleeve 8 during this piercing movement, the return spring 7 is compressed, i.e. tensed. The spring force of the driving spring 6 is stronger than the spring force of the return spring 7 during the entire piercing operation, i.e. including at the start and at the end of the piecing operation. The advantage of this is that the piercing force is reduced.

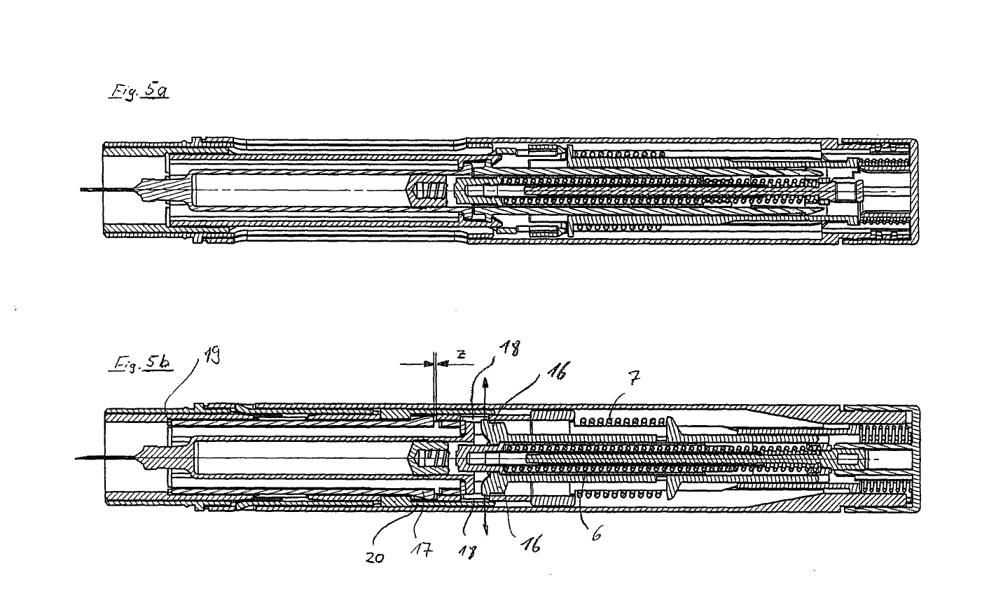

As may be seen from FIGS. 5a and 5b illustrating the situation at the end of the piercing operation, the lock element 16 engages in the cut-out 18 by a movement directed radially outward, as indicated by the arrows in FIG. 5b. To improve this locating action, the lock element 16 has a projection directed radially outward. The lock element 16 fulfils a dual function. As the lock element 16 latches in the cut-out 18, the lock element 16 is simultaneously released from the plunger rod 5 by the movement directed radially outward, releasing the latter for a dispensing movement. Conversely, the movement of the drive structure in the axial direction, i.e. in the proximal direction, is blocked or prevented. As a result of this operation, the driving spring 6 is uncoupled from the return spring 7, i.e. the driving spring 6 has no effect on the tensioning of the return spring 7 in this state. A dispensing movement then follows, during which a clicking noise is emitted at constant times by the signalling unit, which is perceptible to the user of the device.

No additional force due to the piercing operation can be felt by the user of the device. This is absorbed by the snapping action between the operating sleeve 9 and the switch sleeve 8 and is not transmitted to the housing. The force for the piercing operation is directed via the function sleeve 11 to the collar of the product container 2. The piercing operation is therefore forcibly controlled because the function sleeve 11 drives the product container 2 forward until the end of dispensing and the plunger rod 5 is not able to dispense until the lock elements 16 have located in the cut-outs 18. The piercing movement is stopped by the stop 19 on the operating sleeve 9.

During the piercing movement, the switch cam 17 is forced out of the engagement with the cut-out 18 due to the design of the distal boundary of the cut-out 18 of the operating sleeve 9 and is pushed in the distal direction so that it latches in the lock window 20, as illustrated in FIGS. 5a and 5b. The lock element 16 latched in the cut-out 18 is in contact with the proximal boundary of the cut-out 18. Since the lock element 16 and the switch cam 17 are disposed at a defined distance from one another due to their axially fixed arrangement, there is a short distance between the proximal end of the switch cam and the distal end of the lock window 20 when the lock element 16 is engaged by the cut-out, which in this instance is 0.5 to 1 mm, for example. As explained in more detail below, this distance is used to produce a haptic or acoustic signal which is intended to indicate that the product has been fully dispensed. The short distance z results from the difference between the distance existing between the stop surface of the switch cam 17 pointing in the proximal direction and the stop surface pointing in the proximal direction, and the distance of the proximal boundaries of the cut-out 18 and the lock window 20.

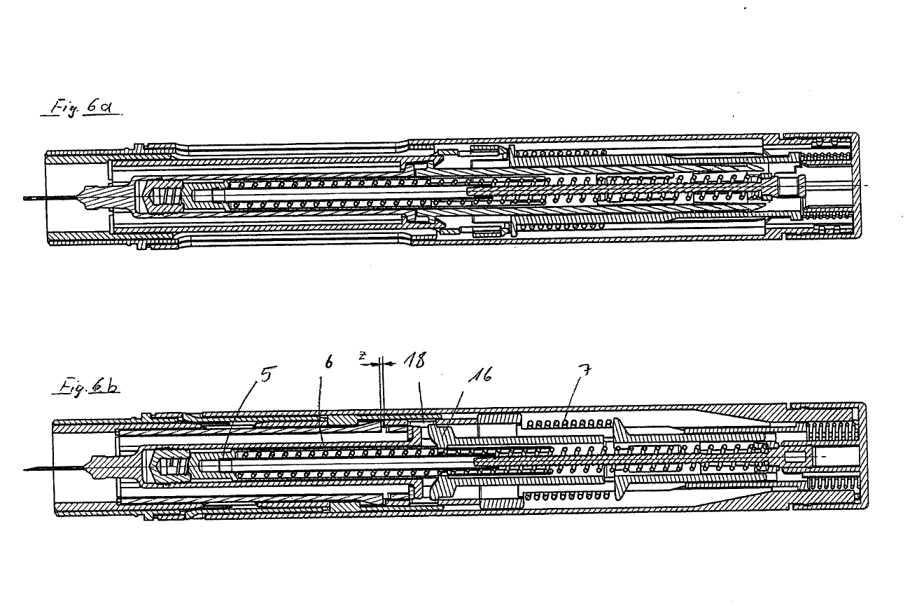

FIGS. 6a and 6b illustrate the injection device in a state in which a product or substance has been dispensed. During dispensing of the product, the external circumferential surface of the sleeve-shaped part of the plunger rod 5 pushes the lock element 16 into the cut-out 18, as a result of which the lock element 16 is locked to prevent it from unlatching from the cut-out 18 while product is being dispensed. The plunger rod 5 may have a cut-out or may be of such a length that when the product has been dispensed, the locking action of the lock element 16 by the external circumferential surface of the plunger rod 5 disappears so that the lock element 16 is able to unlatch from the cut-out 18, as illustrated in FIG. 6b. The unlatching action may be caused by an elastically biased arrangement of the lock element 16 or due to the geometry of the lock element 16, which causes the lock element 16 to be pushed out of the cut-out 18.

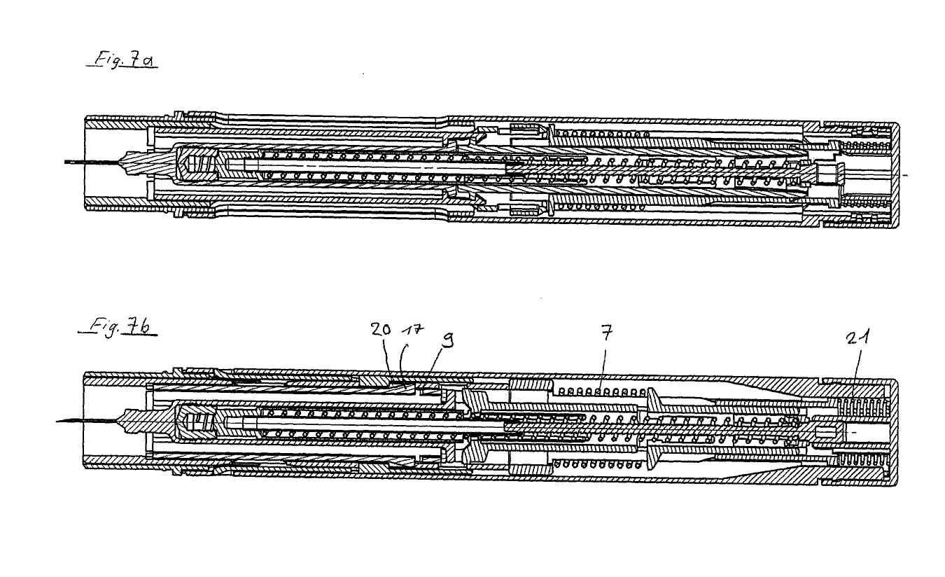

At the end of dispensing the product, the driving spring 6 has further relaxed, while the tensioning of the tensed return spring 7 remains constant. The spring force of the driving spring 6 is now weaker than the spring force of the tensed return spring 7. When the engagement of the lock element 16 with the cut-out 18 is released, the return spring 7 and driving spring 6 are coupled with one another again. As illustrated in FIGS. 7a and 7b, this coupling causes the short distance z (see FIGS. 5b and 6b) to disappear. The drive structure, i.e. in particular the switch cam 17, is moved by its proximal end abruptly onto the distal end of the lock window 20. As the switch cam 17 makes contact, a haptic and/or acoustic signal is generated. This movement by the short distance z does not yet cause the needle 4 to be completely retracted from the patient, however. The patient or user of the device can now wait any time until the needle has been completely pulled out of the patient because he or she can selectively initiate the automatic retraction of the needle of the device.

A complete movement of the needle into the distal end of the housing 1 is still not possible because, as may be seen from FIG. 7b, the switch cam 17 is engaged with the lock window 20 and is thus preventing the spring 7 from relaxing. To release retraction of the needle 4, the user of the device removes the latter from the injection site. As a result, the return spring 21 is able to move the operating sleeve 9 in the distal direction via the switch sleeve 8. As this happens, the drive structure is stationary relative to the operating sleeve 9 so that the switch cam 17 is pushed radially inwardly out of the lock window 20, due to its distal shape, driven by the spring 21 connected to the operating sleeve 9 by the projection 1d. As soon as the switch cam 17 is pushed inward, the needle 4 is free to retract. Also as a result of the releasing action, the return spring 7 is released for a retracting movement. Due to the stronger spring force of the pre-tensed return spring 7, the entire drive structure is pushed or moved in the proximal direction. As a result, the spring 6 is tensed again and the spring force of the return spring 7 is stronger than the spring force of the driving spring 6 during the entire retraction operation, i.e. including up to the end of the retracting movement.

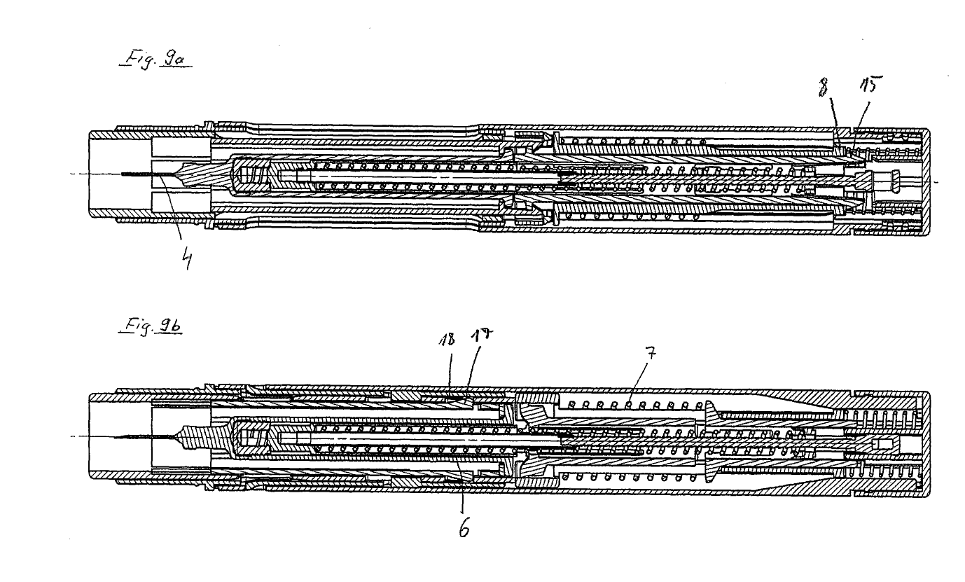

FIGS. 9a and 9b illustrate the injection device in a final state. In this state, the injection device again has the same dimensions it had at the start. Consequently, the cap 32 can be fitted again and the injection device disposed of. In the end position, the needle has been completely retracted into the distal end of the device. The snapper element 15 is latched to the switch sleeve 8 again, as at the start. However, it is not possible to trigger the injection device again because a pre-tensed driving spring 6 would be necessary to do this, as illustrated in FIG. 1a, for example.

In the example of the injection device described above, the piercing and dispensing operation can essentially be triggered by the user with three steps, namely by pulling off the cap and rotating the knob 13 or vice versa, and pressing the distal end of the injection device to the injection site.

FIGS. 10 to 16 illustrate another preferred embodiment of the present invention in the form of an automatic injector. The automatic injector operates in a manner similar to that of the automatic injector illustrated in FIGS. 1a to 9b, but with a few differences with regard to the way in which the piercing operation is triggered. This being the case, those aspects that are different from the automatic injector illustrated in FIGS. 1 to 9 will be described below and reference may be made to the description of the embodiment illustrated in FIGS. 1a to 9b for the other details.

FIG. 10a illustrates the injection device in an initial position, i.e. not triggered and with a cap 32 fitted on the distal end of the injection device. The cap 32 prevents access to the operating sleeve 9, which may be thought of and referred to as the second element within the context of the invention. The cap 32 also may be thought of and referred to as constituting a safety element within the context of the invention. As may be seen from FIG. 10b, the operating sleeve 9, which extends distally beyond the distal end of the housing 1, has a lock element 9a which engages in the housing 1, which may be thought of and referred to as a first element within the context of the invention. The cam-shaped lock element 9a is formed integrally with the operating sleeve 9 on a resilient arm so that it is able to flex transversely to the longitudinal direction L of the injection device. In the arrangement illustrated in FIG. 10b, the lock element 9a locates or is received in the distal end of the housing 1, in the distal front face. In principle, the housing 1 could also have a recess in which the lock element 9a can locate. The cap 32 serving as a safety element has a surface which prevents the lock element 9a from moving out of engagement with the housing 1. The lock element 9a is held in engagement with the housing 1 or is pushed into engagement with the housing 1 by this surface. The cap 32 has a sleeve-shaped portion forming the surface which holds the lock element 9a in engagement with the housing 1. To facilitate the engagement, the surface of the cap 32 is disposed underneath the cam 9a, i.e. the surface is disposed in the path in front of the cam 9a which the cam 9a would follow during an unlatching movement from the housing 1.

In the arrangement illustrated in FIGS. 10a and 10b, the cap 32 is in the secured position in which it blocks the lock element 9a to prevent a movement from or out of the locked position to or into the unlocked position. When removed, e.g. by pulling it off the injection device, the cap 32 is moved into the unsecured position in which it releases the movement of the lock element 9a into the unlocked position, although this does not necessarily mean that the lock element 9a is actually moved into the unlocked position just because the cap 32 has been removed. Instead, another movement may be needed for this purpose, for example a movement of the operating sleeve 9.

FIGS. 11a and 11b illustrate the injection device in a state in which the cap 32 has been removed. In spite of the fact that the cap 32 has been removed, the lock element 9a is still engaged with the housing 1.

Pulling off the cap 32 enables a cap covering the needle 4, which may be made from an elastic material such as rubber or caoutchouc for example, to be removed from the needle 4. To this end, the cap 32 may have a locating member, such as a claw made from plastic or metal, which locates in the needle guard and transmits the movement by which the cap 32 is pulled off to the needle guard. The locating member may be connected to the cap 32 in an axially fixed arrangement.

FIGS. 11a and 11b illustrate the injection device ready for a triggering operation.

By placing and pressing the distal end, e.g. the distal end face of the operating sleeve 9, against the injection site, the operating sleeve 9 is moved in the proximal direction into the housing 1, as a result of which the piercing operation and indirectly also the product dispensing operation are triggered. Due to the movement of the operating sleeve 9 in the proximal direction, the cams 9a resiliently biased radially outward are pushed inwardly, in other words out of the locked position into the unlocked position, as a result of which the movement between the first element and the second element is released. The lock element 9a has a shape, an oblique surface pointing in the proximal direction, which causes the lock element 9a to move out of the locked position into the unlocked position when the operating sleeve 9 is moved relative to the housing 1 in the proximal direction. FIGS. 12a and 12b illustrate the operating sleeve 9 pushed in the proximal direction. As illustrated, the lock element 9a is also in its unlocked position in which it remains pushed radially inward by the inner periphery of the housing 1. The injection device is triggered in the state illustrated in FIGS. 12a and 12b. At the end of the piercing and dispensing operation, the injection device can be removed from the injection site so that the operating sleeve 9 is pushed in the distal direction relative to the housing 1 by the return spring 21, so that the cap 32 can refitted in readiness for disposal together with the injection device, although this step is not absolutely necessary.

FIGS. 10a to 16 illustrate a safety element in the form of an activator lock 14. The purpose of the activator lock 14, which can be understood from FIG. 15, is to hold the snapper elements 15 serving as a lock element in the context of the invention in engagement with the switch sleeve 8, which may be regarded as a first element of the invention. This engagement may be thought of and referred to as a locked position because a movement of the function sleeve 11 serving as a second element relative to the switch sleeve 8 is prevented due to the fact that the snapper elements 15 are located in the switch sleeve 8. The activator lock 14 supports the snapper element 15 radially from inside and thus prevents the snapper element 15 from unlatching from the switch sleeve 8, into an unlocked position in which the function sleeve 11 is able to move in the distal direction relative to the switch sleeve 8. The activator lock 14 is connected to the switch sleeve 8 so that it is able to rotate and axially fixed with it. As may be seen from FIGS. 10a and 16, the switch sleeve 8 has a web 8a, in which the activator lock 14 latches in a positive fit so that it is able to rotate relative to the switch sleeve 8, at least by an angle α. When the operating sleeve 9 is operated, the activator lock 14 can be displaced in conjunction with the switch sleeve 8. The activator lock 14 remains on the same axial level as the snapper element 15 during triggering and until the end of triggering. The snapper element 15 is moved together with the switch sleeve 8 in the proximal direction during the triggering movement of the operating sleeve 9. During the triggering movement, i.e. the movement of the switch sleeve 8 in the proximal direction from the secured position illustrated in FIGS. 10a, 11a and 13a into an unsecured position, the activator lock 14 can be rotated by an angle α. Due to the rotation of the activator lock 14, the snapper element 15 is released so that it can move out of the locked position into an unlocked position. The snapper element 15 can be moved into the unlocked position due to the fact that the snapper elements 15 are released from the engagement with the switch sleeve 8 by their biased pre-tensioning when no longer supported by the activator lock 14. As illustrated in FIG. 12a, the snapper element 15 remains engaged with the switch sleeve 8 when the activator lock 14 is moved out of its secured position. The snapper element 15 is able to engage with the housing 1 in such a way that it can be moved out of the locked position into the unlocked position specifically when the operating sleeve 9 is operated. To this end, the housing may have an activator cam 13a, which is axially aligned with the snapper element 15 in the operating direction so that the snapper element 15 moves into engagement with the activator cam 13a when the operating sleeve 9 is operated, as a result of which the axial movement of the snapper element 15 is converted into a movement directed transversely to the longitudinal axis L. In the proximal direction, the snapper element 15 may have an inclined surface which slides down the activator cam 13a during an axial movement and thus forces the snapper element 15 in the direction of the mid-axis L, into the unlocked position. When the snapper element 15 is in the unlocked position, the function sleeve 11 can be pushed in the distal direction, thereby resulting in the piercing action of the needle 4.

When the operating sleeve 9 is operated, the activator lock 14 is able to move into engagement with the housing 1 so that the axial movement of the activator lock 14 is converted into a rotating movement. To this end, the housing 1 may have a guide element 13b, on which the activator lock 14 slides during its axial movement, and is caused into a rotating movement. The guide element 13b is disposed so that when the operating sleeve 9 is operated, the activator lock 14 is firstly rotated out of the secured position into the unsecured position and then the snapper element 15 is moved out of the locked position into the unlocked position by engaging with the activator cam 13a. To make it easier for the activator lock 14 to slide on the guide element 13, the guide element 13b and/or the activator lock 14 may have an oblique surface 14a. The oblique surfaces may be inclined in the circumferential direction, as illustrated in FIGS. 13 to 15 for example.

As a result of this arrangement of the activator lock 14, the user triggers the device in two steps and the device can not be inadvertently triggered during handling and transport. Triggering involves the steps of pulling off the cap and pressing the device to the injection point by its distal end. These movements are motor movements which can also be effected by persons suffering from difficulty in performing fine motor movements.

In FIG. 15, the activator lock 14 is axially secured relative to the switch sleeve 8 due to the fact that the web or socket 8a of the switch sleeve 8 is held between a forked portion 14d and a collar 14b with a slight axial clearance. The activator lock 14 has a shaft from which two wings project in a T-shape, which are able to hold the snapper elements 15 in their locked position. A purpose of the forked portion 14d of the activator lock 14 is to ensure that the activator lock 14 is able to latch positively in the cut-out in the switch sleeve 8 provided for it. The activator lock 14 has at least one projection 14c, around the circumference of the shaft. The projection 14c may selectively locate in the cut-out 8c and 8d of the switch sleeve 8, depending on the position of angular rotation. This prevents any undesired turning of the activator lock 14 such as might be caused by vibrations. Instead, the engagement with the housing 1, for example, is necessary to cause a turning movement of the activator lock 14. The cut-out 8d in which the projection 14c locates when the activator lock 14 is in its unsecured position is offset by the angle α from the cut-out 8c in which the projection 14c is disposed when the activator lock 14 is in the secured position. The angle is an angle of less than 90°, for example 45°+/−10°, but angles bigger than 90° but smaller than 180° would also be possible. The projection 14c latches in or out of the cut-outs 8c, 8d due to the elasticity of the material of the projection 14c and the switch sleeve 8.

FIG. 16 illustrates the switch sleeve 8 with its distal end pointing toward the observer. The switch sleeve 8 has cut-outs 8b through which the arms of the function sleeve 11 on which the lock elements 15 are disposed can extend.

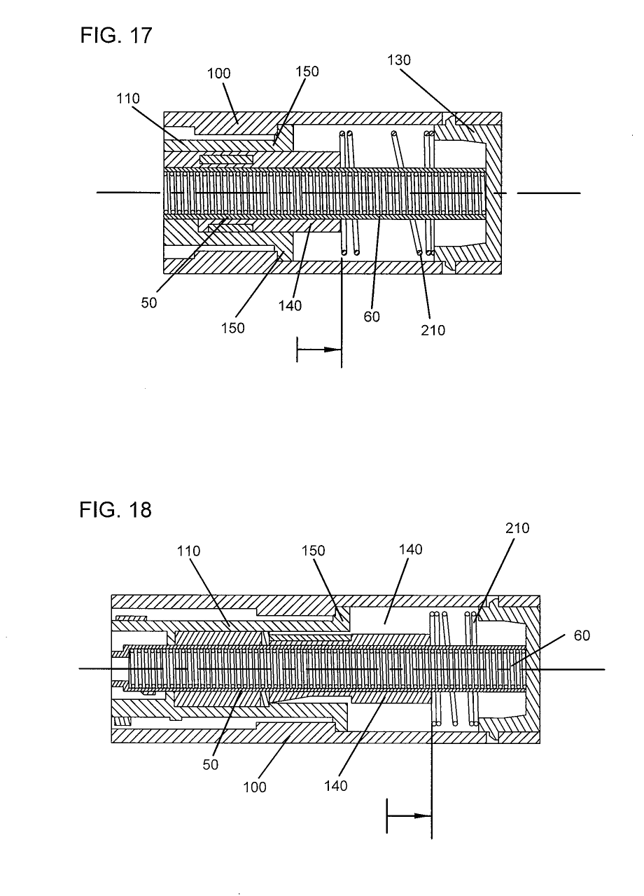

FIGS. 17 and 18 illustrate another embodiment of the present invention. The device comprises a housing 100, on the proximal end of which a closure element 130 is connected to the housing 100 so that it can not move axially and/or not rotate. The closure element 130 may therefore be regarded as part of the housing 100. A piercing and/or dispensing spring 60 is supported by its proximal end on the closure element 130 and by its distal end on a hollow plunger rod 50, in which the spring 60 is accommodated. In the state illustrated in FIGS. 17 and 18, the spring 60 is pre-tensioned with the energy needed to perform the piercing and dispensing operation. The plunger rod 50 is retained by a function sleeve 110. The function sleeve 110 has a snapper element 150, which engages with an inwardly extending projection of the housing 100. The snapper element 150 may be regarded as a lock element, the housing 100 as a first element and the function sleeve 110 as a second element within the context of the invention. The device also has an activator lock 140 serving as a safety element which, as illustrated in FIG. 17, is disposed at the same axial level as the snapper element 150 in an initial state. As a result, the snapper element 150 is prevented from moving out of engagement with the housing 100, which corresponds to the locked position, into an unlocked position in which the snapper element 150 is no longer engaged with the housing 100. The activator lock 140 is connected to an operating element, such as an operating sleeve as illustrated in FIGS. 10a, 10b, so as to be axially immobile. When the injection device is applied to the injection site, the activator lock 140 is pushed in the distal direction (arrow), i.e. out of a secured position in which the snapper element 150 is prevented from moving out of the locked position into the unlocked position, into an unsecured position in which the movement of the lock element 150 into the unlocked position is released. As may be seen from FIG. 18, the activator lock 140 is no longer on the same axial level as the snapper element 150 and the snapper element is therefore able to effect a movement into the unlocked position. The snapper element 150 and/or the housing 100 are shaped or have inclined surfaces which cause the snapper element 150 to slide on the housing 100 due to the force of the pre-tensioned spring 60 which is transmitted via the plunger rod 50 to the function sleeve 110, and thus effect a movement radially inwardly into the unlocked position. As a result the function sleeve 110 is released and can effect a movement relative to the housing 100 driven by the spring 60. Coupled with the function sleeve 110 is a product container, which is moved in the distal direction by the movement of the function sleeve 110, causing a needle mounted on the product container to be injected into the patient. The plunger rod 50 is then released so that it can effect a dispensing movement. The reference numbers used for parts of the injection device illustrated in FIGS. 17 and 18 correspond to the reference numbers used for FIGS. 1a to 16, but the reference numbers used for FIGS. 17 and 18 are each suffixed by a zero. For more details with respect to FIGS. 17 and 18, reference may be made to the corresponding parts of the description given in connection with FIGS. 1 to 16.

Embodiments of the present invention, including preferred embodiments, have been presented for the purpose of illustration and description. They are not intended to be exhaustive or to limit the invention to the precise forms and steps disclosed. The embodiments were chosen and described to illustrate the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for administering a medicinal substance, the device comprising a first element and a second element, wherein at least one of the first and second elements is moveable relative to the other, a lock element moveable to and from a locked position in which it prevents the relative movement of the at least one of the first and second elements and an unlocked position in which it permits the relative movement of the at least one of the first and second elements, and a safety element moveable to and from a secured position in which it prevents the lock element from moving from the locked position to the unlocked position and an unsecured position in which it permits the movement of the lock element to the unlocked position.

2. The device as claimed in claim 1, wherein the first element is associated with a housing of the device and the second element is one of an operating mechanism or a drive member of the device which comprises a drive means for supplying the energy for at least one of a piercing movement of a needle associated with the device and an administering of the substance.

3. The device as claimed in claim 2, wherein the safety element can be moved out of the secured position into the unsecured position before or during operation of the operating mechanism or drive member.

4. A device for administering a medicinal product, the device comprising:
a) a first element,
b) a second element moveable relative to the first element,
c) a lock element moveable from a locked position, in which it prevents a movement between the first element and the second element, to an unlocked position, in which it permits the movement between the first element and the second element, and
d) a safety element moveable from a secured position, in which it blocks the lock element to prevent the movement out of the locked position into the unlocked position, to an unsecured position, in which it permits the movement of the lock element into the unlocked position.

5. The device as claimed in claim 4, wherein the lock element is one of a separate part or a part formed on the first element or second element

6. The device as claimed in claim 4, wherein the lock element is moveable from the locked position to the unlocked position by a movement directed transversely to the longitudinal length of the injection device.

7. The device as claimed in claim 4, wherein the safety element is arranged on the same level as the lock element in the longitudinal direction in its secured position.

8. The device as claimed in claim 4, wherein the lock element engages in at least one of the first element and second element in the locked position and the safety element holds the lock element in engagement in its secured position.

9. The device as claimed in claim 8, wherein the lock element is arranged so that it tends to either move out of the locked engagement or remain in the locked engagement when the safety element is moved out of the secured position.

10. The device as claimed in claim 9, wherein the second element is arranged inside the first element, the safety element is arranged inside the first element and the lock element moveable out of the locked engagement by a movement directed toward a mid-axis of the device.

11. The device as claimed in claim 4, wherein the safety element is moveable out of the secured position into the unsecured position by one of a rotating movement or an axial movement.

12. The device as claimed in claim 4, wherein the safety element is removeable from the injection device.

13. The device as claimed in claim 12, wherein the safety element comprises a removable cap.

14. The device as claimed in claim 4, wherein the first element is a housing and the second element is one of an operating mechanism or a drive member which comprises a drive means for supplying the energy for at least one of a piercing movement of a needle and a movement to dispense the product in a tensed state.

15. The device as claimed in claim 14, wherein the safety element is moveable out of the secured position into an unsecured position before or during operation of an operating element by which at least one of a piercing operation and a dispensing operation can be triggered.

16. The device as claimed in claim 15, wherein the safety element has a position lock which holds the safety element in one of a secured position and unsecured position by means of at least one of a positive and non-positive engagement.

17. A method of triggering an injection device, comprising:
a) moving a safety element (32; 14; 140) from a secured position, in which it prevents a lock element (9a; 15; 150) from moving out of the locked position into the unlocked position, into an unsecured position, in which it permits the movement of the lock element (9a; 15; 150) into the unlocked position,
b) moving a lock element (9a; 15; 150) out of a locked position, in which it prevents a movement between a first element (1; 8; 100) and a second element (9; 11; 110), into an unlocked position, in which it permits the movement between the first element (1; 8; 100) and the second element (9; 11; 110),
c) moving a second element (9; 11; 100) relative to the first element (1 8; 100).
